(19) United States
(12) Patent Application Publication
(10) Pub. No.: US 2010/0305315 A1
(43) Pub. Date: Dec. 2, 2010

(54) Process for the preparation of 2-(primary/secondary amino)hydrocarbyl)-carbamoyl-7-oxo-2,6-diaza-bicyclo [3.2.0.]heptane-6-sulfonic acid derivatives

(75) Inventors: Eric Desarbe, Mulhouse (FR); Florian Richalet, Huningue (FR)

Correspondence Address: GIBBONS P.C., ONE GATEWAY CENTER, NEWARK, NJ, 07102, US

(73) Assignee: Basilea Pharmaceutica AG

(21) Appl. No.: 12/744,565

(22) PCT Filed: Dec. 4, 2008
(86) PCT No.: PCT/EP08/66826

§ 371 Date: Jul. 1, 2010

(30) Foreign Application Priority Data
Dec. 4, 2007 (EP) 07122236.8

(51) Int. Cl. C07D 487/04

(52) U.S. Cl. 540/203

(57) ABSTRACT

A process for the production of a compound of formula (I) wherein ^{A}LINKER^{B} represents a linker moiety of formula (V): ^{A}[G1-G2*-G3]^{B}; wherein A and B indicate the orientation of the group of formula (V) in formula (I); G1, G2 and G3 have specific meanings described herein and may be present or absent, with the proviso that at least one of G1 or G3 is present; which linker group may furthermore optionally contain one or more groups of formula (VI); and/or other substituents; and R1 represents hydrogen or a C₁-C₄-alkyl group; R2 represents hydrogen or a C₁-C₄-alkyl group; R3 independently at each occurrence, represents hydrogen or a C₁-C₄-alkyl group; x is 0 or 1; y is 0 or 1; z independently at each occurrence, is 0 or 1; and (—) represents a single bond between a primary, secondary or tertiary carbon atom of the moiety ^{A}LINKER^{B} and the adjacent nitrogen atom; in which process (A) a compound of formula (II) is reacted with a compound of formula (III) wherein Pr represents an amino protecting group selected from t-butyloxy carbonyl (t-Boc), 1-methyl-1-(4-biphenylyl)ethyloxy carbonyl (Bpoc), 1-(1-adamantyl)-1-methylethyloxy carbonyl (Adpoc), 1-(3,5-di-t-butylphenyl)-1-methylethyloxy carbonyl (t-Bumeoc), 1-adamantyloxy carbonyl (Adoc), p-methoxybenzyloxy carbonyl (Moz), o,p-dimethoxybenzyloxy carbonyl, ^{A}LINKER^{B} has the same meaning as in formula (I) with the exception that one or more of the optional groups of formula (VII) may be replaced by a group of formula (VII) and R1; R2; R3; x; y; z and (—), at each occurrence, have the same meaning as in formula (I) and Pr is as defined above; in a dipolar aprotic solvent in the presence of a base to obtain a compound of formula (IV) wherein Pr; ^{A}LINKER^{B}; R1; R2; R3; x; y; z; and (—), at each occurrence, have the same meaning as in formula (III); which compound is then (B) deprotected by reaction with formic acid or a mixture of formic acid or acetic acid with hydrochloric acid or hydrobromic acid, to give the compound of formula (I) as well as the compounds of the aforementioned formula (IV).

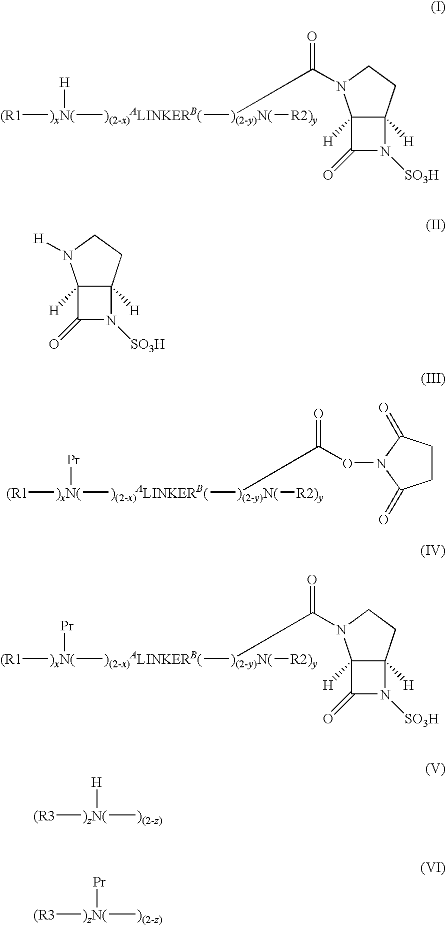

The present invention relates to the process for the manufacture of compounds of general formula (I):

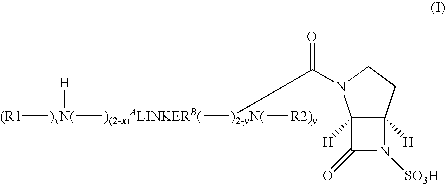

wherein
^{A}LINKER^{B} represents a linker moiety of formula

^{A}[G1-G2*-G3]^{B} (V)
wherein
^{A} and ^{B} indicate the orientation of the group of formula (V) in formula (I);
G1, G2 or G3 may be present or absent, with the proviso that at least one of G1 or G3 is present; and,
G1 if x is 1 and y is 1 and both, G2 and G3, are absent or if x is 1 and G2 and/or G3 are present, represents a C₆-C₁₄arylene; a saturated or non-aromatic unsaturated C₃-C₉cycloalkylene or a saturated or unsaturated heterocyclodiyl group comprising 5 to 9 ring atoms containing one or more heteroatoms selected from N, O and S, which groups may be unsubstituted or substituted or,
   if x is 0 and y is 1 and both, G2 and G3, are absent or if x is 0 and G2 and/or G3 are present, together with the group

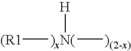

forms a heterocyclyl group comprising 5 to 9 ring atoms containing one or more heteroatoms selected from N, O and S, which may be unsubstituted or substituted; or,
if x is 0 and y is 0 and both, G2 and G3, are absent, together with the groups

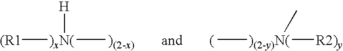

forms a heterocyclyl group comprising 5 to 9 ring atoms containing two or more heteroatoms selected from N, O and S, which may be unsubstituted or substituted;
G2 represents a group selected from
   —(CₙH₂ₙ)—; —(NH)—; —(C═O)—; —(C═O)NH—*; —HN(C═O)—*;
   —HN(C═O)NH—; —(CₙH₂ₙ)—[X—CₙH₂ₙ]ₘ—(C═O)NH—*;
   —HN(C═O)—[CₙH₂ₙ]—X]ₘ—(CₙH₂ₙ)—*;
   —(CₙH₂ₙ)—[X—CₙH₂ₙ]ₘ—HN(C═O)—*;
   —(O═C)NH—[CₙH₂ₙ]—X]ₘ—(CₙH₂ₙ)—*;
   —(CₙH₂ₙ)—[X—CₙH₂ₙ]ₘ—HN(C═O)NH—*,
   and
   —HN(C═O)NH—(CₙH₂ₙ)—[X—CₙH₂ₙ]ₘ—*
   wherein
   * indicates the bond of G2 indicated in formula (V) with * and X represents, independently at each occurrence,
   —O—; —NH—; —(C═O)NH—; —NH(C═O)—; —(C═O)O—; —O(C═O)—;
   —NH(C═O)NH—; —NH(C═O)O— or —O(C═O)NH—
   n is, independently at each occurrence, 1, 2, 3 or 4, in particular 2; and
   m is 0, 1, 2 or 3, in particular 0 or 1; and,
G3 if y is 1 and G1 and/or G2 are present, represents a C₆-C₁₄arylene; a saturated or non-aromatic unsaturated C₃-C₉cycloalkylene or a saturated or unsaturated heterocyclodiyl group comprising 5 to 9 ring atoms containing one or more heteroatoms selected from N, O and S, which groups may be unsubstituted or substituted, or,
   if y is 0 and x is 1 and both, G1 and G2, are absent or if y is 0 and G1 and/or G2 are present, together with the group

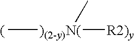

forms a heterocyclyl group comprising 5 to 9 ring atoms containing one or more heteroatoms selected from N, O and S, which may be unsubstituted or substituted,
which linker group may furthermore optionally contain one or more groups of formula:

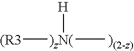

and/or other substituents;
R1 represents hydrogen or a C₁-C₄-alkyl group;
R2 represents hydrogen or a C₁-C₄-alkyl group;
R3 independently at each occurrence, represents hydrogen or a C₁-C₄-alkyl group;
x is 0 or 1;
y is 0 or 1;
z independently at each occurrence, is 0 or 1; and
(—) represents a single bond between a primary, secondary or tertiary carbon atom of the moiety ^{A}LINKER^{B} and the adjacent nitrogen atom.

Compounds of formula (I) and their manufacture are known in the art and described e.g. in WO 2007/065288 A2; J. Med. Chem. 1988, 3961 or WO2008/039420, the contents of which is considered to be explicitly incorporated in the present application.

J. Med. Chem. 1988, 3961-3971 discloses the reaction of compounds of formula (II) with benzyl-protected derivatives of formula (III') to give the compounds of formula (IV′) (cf. Scheme 1 below). These compounds are then deprotected by hydrogenation over Pd/C in the presence of HCl at 50° C., the catalyst is filtered off and the product is then purified.

WO2008/039420 discloses the reaction of compounds of formula II with benzyloxycarbonyl-protected derivatives of formula III′ to give the compounds of formula IV′. These compounds are then deprotected by hydrogenation over Pd/C, the catalyst is filtered off and the product is then purified.

The deprotection with Pd as catalyst however has the disadvantage, in particular if applied at a very late stage of the manufacturing process, that it is difficult to remove the Pd from the final product to an extent required by the pharmaceutical regulatory authorities. It is therefore often necessary to carry out elaborate and complex purification steps for sufficiently reducing the Pd level of the API to comply with the legal requirements.

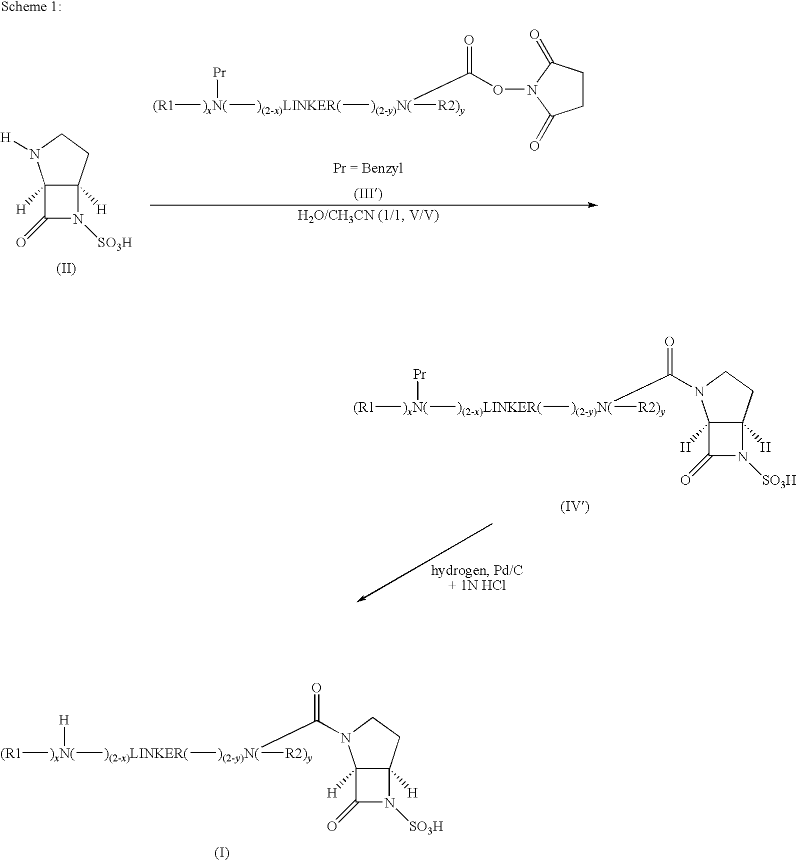

Different acids can also be used for removing certain amino protecting groups under acidic conditions. Trifluoroacetic acid and HCl are mainly employed for this purpose. A corresponding conventional large-scale manufacturing process for compounds of formula (I) uses a synthesis starting from (1S,5R)-7-oxo-2,6-diazabicyclo[3.2.0]heptane-6-sulfonic acid (II) and reacts said compound with appropriate succinimidyl derivatives of formula (A). The terminal amino group attached to the LINKER of the compound of formula (A) is not protected with an amino protecting group during the reaction (Scheme 2).

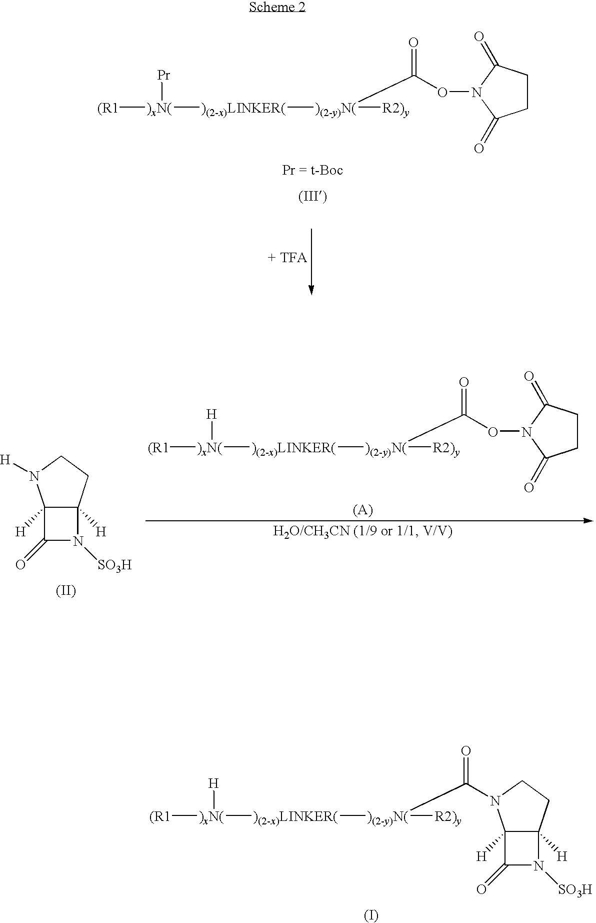

The deprotection of the compound (III″) before reacting it with the compound (II) is necessary when working with usual methods like reacting the compound (IV) with TFA (trifluoroacetic acid) or aqueous HCl according to Scheme 2 because a deprotection with these acids results in a considerable degradation of the product (I). The product can therefore not be used in the further process as it is but has to be purified before in order to ensure the production of sufficiently pure reaction product of formula (I). This purification can only be carried out using chromatographic methods which are generally not convenient in large scale production (e.g large quantities of silica gel and the evaporation of large solvent volumes are required). Furthermore TFA is generally considered to be harmful, its inhalation and accordingly its release into the environment can be dangerous.

The necessity of using the unprotected compound A as in Scheme 2 has furthermore the disadvantage that compound A has to be used at a considerable excess, usually at about 40 or even more mole percent to compensate for losses of said compound caused by side reactions of compound (A) with itself. The required excess of compound A increases the production costs, and the side products obtained are an additional source of impurities for the end product and have to be removed.

For the similar reasons it is also not possible in usual industrial practice to react the compound of formula (II) directly with the compound (III′) and to deprotect the reaction product conventionally with TFA or HCl because this deprotection would again result in a rather strong degradation of the end product, and therefore again require an elaborate and complex purification of the product which is not really suitably for large scale production.

Another significant disadvantage of the prior art manufacturing process for the compounds of formula (I), in particular in view of a large scale production, is the solvent system which is a mixture of acetonitrile and water. The solubility of (1S,5R)-7-oxo-2,6-diaza-bicyclo[3.2.0]heptane-6-sulfonic acid (II) in such mixtures is low, e.g. less than 10 mg per mL in the 9:1 (V/V) mixture used according to WO 2007/065288 A2 and even worse in a 1:1 (V/V) mixture as used according to J. Med. Chem. 1988, 3961. This low solubility results in a need for large solvent volumes which in turn require the use of devices being unacceptable large in volume.

In summary, it is difficult to apply the prior art methods for manufacturing compounds of formula (I) in large scale production processes for such compounds. These compounds, however, attract more and more attention as potential active pharmaceutical ingredients so that an effective and economical manufacturing process for compounds of formula (I) is desirable.

It has now been found, that a novel two step manufacturing process for compounds of formula (I) starting from (1S,5R)-7-oxo-2,6-diaza-bicyclo[3.2.0]-heptane-6-sulfonic acid avoids defects of the known prior art process including the aforementioned defects and can readily be used for large scale manufacturing of compounds of formula (I). This novel process uses in particular selected amino protecting groups in combination with selected acids for a gentle deprotection of the protected (terminal) amino groups.

Accordingly, the invention relates to a process for the production of a compound of formula (I)

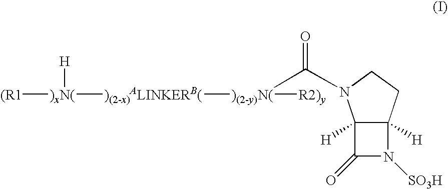

wherein
^{A}LINKER^{B} represents a linker moiety of formula

^{A}[G1-G2*-G3]^{B} (V)
wherein
^{A} and ^{B} indicate the orientation of the group of formula (V) in formula (I);
G1, G2 or G3 may be present or absent, with the proviso that at least one of G1 or G3 is present; and,
G1 if x is 1 and y is 1 and both, G2 and G3, are absent or if x is 1 and G2 and/or G3 are present, represents a C₆-C₁₄arylene; a saturated or non-aromatic unsaturated C₃-C₉cycloalkylene or a saturated or unsaturated heterocyclodiyl group comprising 5 to 9-ring atoms containing one or more heteroatoms selected from N, O and S, which groups may be unsubstituted or substituted or,
   if x is 0 and y is 1 and both, G2 and G3, are absent or if x is 0 and G2 and/or G3 are present, together with the group

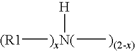

forms a heterocyclyl group comprising 5 to 9 ring atoms containing one or more heteroatoms selected from N, O and S, which may be unsubstituted or substituted; or,
if x is 0 and y is 0 and both, G2 and G3, are absent, together with the groups

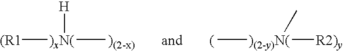

forms a heterocyclyl group comprising 5 to 9 ring atoms containing two or more heteroatoms selected from N, O and S, which may be unsubstituted or substituted;
G2 represents a group selected from
   —(CₙH₂ₙ)—; —(NH)—; —(C═O)—; —(C═O)NH—*; —HN(C═O)—*;
   —HN(C═O)NH—; —(CₙH₂ₙ)—[X—CₙH₂ₙ]ₘ—(C═O)NH—*;
   —HN(C═O)—[CₙH₂ₙ]—X]ₘ—(CₙH₂ₙ)—*;
   —(CₙH₂ₙ)—[X—CₙH₂ₙ]ₘ—HN(C═O)—*;
   —(O═C)NH—[CₙH₂ₙ]—X]ₘ—(CₙH₂ₙ)—*;
   —(CₙH₂ₙ)—[X—CₙH₂ₙ]ₘ—HN(C═O)NH—*,
   and
   —HN(C═O)NH—(CₙH₂ₙ)—[X—CₙH₂ₙ]ₘ—*
   wherein
   * indicates the bond of G2 indicated in formula (V) with * and X represents, independently at each occurrence,
   —O—; —NH—; —(C═O)NH—; —NH(C═O)—; —(C═O)O—; —O(C═O)—;
   —NH(C═O)NH—; —NH(C═O)O— or —O(C═O)NH—
   n is, independently at each occurrence, 1, 2, 3 or 4, in particular 2; and
   m is 0, 1, 2 or 3, in particular 0 or 1; and,
G3 if y is 1 and G1 and/or G2 are present, represents a C₆-C₁₄arylene; a saturated or non-aromatic unsaturated C₃-C₉cycloalkylene or a saturated or unsaturated heterocyclodiyl group comprising 5 to 9 ring atoms containing one or more heteroatoms selected from N, O and S, which groups may be unsubstituted or substituted, or,
   if y is 0 and x is 1 and both, G1 and G2, are absent or if y is 0 and G1 and/or G2 are present, together with the group

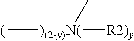

forms a heterocyclyl group comprising 5 to 9 ring atoms containing one or more heteroatoms selected from N, O and S, which may be unsubstituted or substituted,
which linker group may furthermore optionally contain one or more groups of formula:

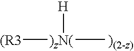

and/or other substituents;
R1 represents hydrogen or a C₁-C₄-alkyl group;
R2 represents hydrogen or a C₁-C₄-alkyl group;
R3 independently at each occurrence, represents hydrogen or a C₁-C₄-alkyl group;
x is 0 or 1;
y is 0 or 1;
z independently at each occurrence, is 0 or 1; and
(—) represents a single bond between a primary, secondary or tertiary carbon atom of the moiety ^{A}LINKER^{B} and the adjacent nitrogen atom in which process
(A) a compound of formula (II)

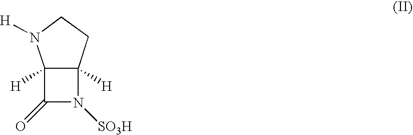

is reacted with a compound of formula (III)

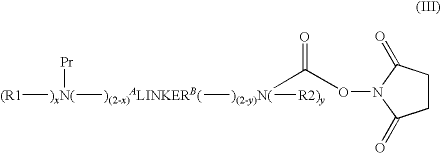

wherein
Pr represents an amino protecting group selected from t-butyloxy carbonyl (t-Boc), 1-methyl-1-(4-biphenylyl)ethyloxy carbonyl (Bpoc), 1-(1-adamantyl)-1-methylethyloxy carbonyl (Adpoc), 1-(3,5-di-t-butylphenyl)-1-methylethyloxy carbonyl (t-Bumeoc), 1-adamantyloxy carbonyl (Adoc), p-methoxybenzyloxy carbonyl (Moz), and o,p-dimethoxybenzyloxy carbonyl;
^{A}LINKER^{B} has the same meaning as in formula (I) with the exception that one or more of the optional groups of formula:

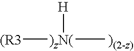

may be replaced by a group of formula:

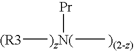

and
R1; R2; R3; x; y; z and (—), at each occurrence, have the same meaning as in formula (I) and Pr is as defined above;
in a dipolar aprotic solvent in the presence of a base to obtain a compound of formula (IV)

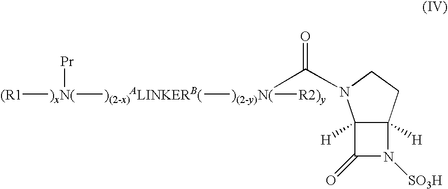

wherein
Pr; ^{A}LINKER^{B}; R1; R2; R3; x; y; z; and (—), at each occurrence, have the same meaning as in formula (III);
which compound is then

(B) deprotected by reaction with formic acid or a mixture of formic acid or acetic acid with hydrochloric acid or hydrobromic acid, to give the compound of formula (I).

For the purposes of the present application the term “^{A}LINKER^{B}” is generally understood to include moieties consisting of a single group of atoms as defined in claim 1. This is the case when at least one of x or y is 1, in which case the moiety ^{A}LINKER^{B} represents a (4-x-y)-valent moiety, i.e. either a 2-valent (x and y are 1) or 3-valent (one of x and y is 0) moiety. If x and y are simultaneously 0, however, LINKER can represent a coherent 4-valent moiety, but can also represent an assembly of two atom groups which are not linked together by a chemical bond. By way of example, if the moiety:

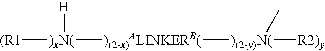

in formula (I) represents the group:

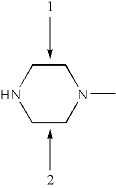

then ^{A}LINKER^{B} defines an assembly of two independent ethylene groups 1 and 2, both of which link the two nitrogen atoms of the group:

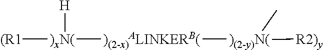

thereby forming a piperazinyl group.

For the purposes of the present application a “primary carbon atom” is meant to be a carbon atom which is linked to one further carbon atom, all other atoms linked to said primary carbon atom being hydrogen or non-carbon atoms. Similarly a “secondary carbon atom” is meant to be a carbon atom linked to two further carbon atoms and a “tertiary carbon atom” a carbon atom linked to three further carbon atoms. Preferably, (—) represents a single bond between a primary, or a secondary carbon atom of the moiety ^{A}LINKER^{B} and the adjacent nitrogen atom

Preferably,
^{A}LINKER^{B} represents a linker moiety of formula

^{A}[G1-G2*-G3]^{B} (V)
wherein
^{A} and ^{B} indicate the orientation of the group of formula (V) in formula (I);
G1, G2 or G3 may be present or absent, with the proviso that at least one of G1 or G3 is present; and,
G1 if x is 1 and y is 1 and both, G2 and G3, are absent or if x is 1 and G2 and/or G3 are present, represents a C₆-C₁₄arylene; a saturated or non-aromatic unsaturated C₃-C₇cycloalkylene or a saturated or unsaturated heterocyclodiyl group comprising 5 to 7 ring atoms containing one or more heteroatoms selected from N, O and S, which groups may be unsubstituted or substituted or,
   if x is 0 and y is 1 and both, G2 and G3, are absent or if x is 0 and G2 and/or G3 are present, together with the group

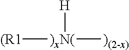

forms a heterocyclyl group comprising 5 to 7 ring atoms containing one or more heteroatoms selected from N, O and S, which may be unsubstituted or substituted; or,
if x is 0 and y is 0 and both, G2 and G3, are absent, together with the groups

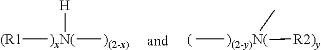

forms a heterocyclyl group comprising 5 to 7 ring atoms containing two or more heteroatoms selected from N, O and S, which may be unsubstituted or substituted;
G2 represents a group selected from
   —(CₙH₂ₙ)—; —(NH)—; —(C═O)—; —(C═O)NH—*; —HN(C═O)—*;
   —HN(C═O)NH—; —(CₙH₂ₙ)—[X—CₙH₂ₙ]ₘ—(C═O)NH—*
   —(CₙH₂ₙ)—[X—CₙH₂ₙ]ₘ—HN(C═O)—*, and
   —(CₙH₂ₙ)—[X—CₙH₂ₙ]ₘ—HN(C═O)NH—*,
   wherein
   * indicates the bond of G2 indicated in formula (V) with * and X represents, independently at each occurrence,
   —O—; —NH—; —(C═O)NH—; —NH(C═O)—; —(C═O)O—; —O(C═O)—;
   —NH(C═O)NH—; —NH(C═O)O— or —O(C═O)NH—
   n is, independently at each occurrence, 1, 2, 3 or 4, in particular 2; and
   m is 0, 1, 2 or 3, in particular 0 or 1; and,
G3 if y is 1 and G1 and/or G2 are present, represents a C₆-C₁₄arylene; a saturated or non-aromatic unsaturated C₃-C₇cycloalkylene or a saturated or unsaturated heterocyclodiyl group comprising 5 to 7 ring atoms containing one or more heteroatoms selected from N, O and S, which groups may be unsubstituted or substituted, or,
   if y is 0 and x is 1 and both, G1 and G2, are absent or if y is 0 and G1 and/or G2 are present, together with the group

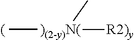

forms a heterocyclyl group comprising 5 to 7 ring atoms containing one or more heteroatoms selected from N, O and S, which may be unsubstituted or substituted,
which linker group may furthermore optionally contain one or more groups of formula:

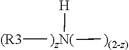

and/or other substituents.

The process of the present invention is particularly useful for the manufacture of compounds of formula (I) wherein the moiety ^{A}LINKER^{B} is free from optional groups of formula:

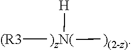

Furthermore, compounds of formula (I) are preferred wherein y is 1, in particular when R2 is hydrogen. It is preferred as well when R1 in formula (I) represents hydrogen or x is 0.

Suitable substituents of the moiety ^{A}LINKER^{B} or the groups G1 and/or G3 include beside of the already mentioned groups of formula:

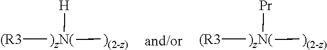

wherein R3, z and Pr have the already mentioned meaning, e.g. C₁-C₆alkyl, preferably C₁-C₄alkyl; hydroxy, C₁-C₆alkoxy, preferably C₁-C₄alkoxy; halogen, in particular fluoro, chloro, and bromo; —(CH₂)ᵤCN, —(CH₂)ᵤN(R4)₂; —(CH₂)ᵤC(O)N(R4)₂, —(CH₂)ᵤSO₂N(R4)₂, —(CH₂)ᵤCO₂R4, —(CH₂)ᵤC(O)R4, —(CH₂)ᵤOC(O)R4, —(CH₂)ᵤNHC(O)R4, —(CH₂)ᵤNHC(O)₂R4, —(CH₂)ᵤNHSO₂R4, —(CH₂)ᵤC(═NH)NH₂, —(CH₂)ᵤC(═NH)H and the like, wherein each

u is independently 0, 1, 2, 3 or 4 and each

R4 is independently hydrogen or C₁-C₄alkyl.

Preferred embodiments of the compounds of formula (I) include the corresponding compounds wherein
^{A}LINKER^{B} represents a moiety of formula
   G1 and x is 0 or 1 and y is 1, or x and y are both 0;
   G3 and y is 0 or 1;
   [G2*-G3] and x and y are both 1; or
   [G1-G2*-G3] and x and y are, independently of one another, 0 or 1, and
G1, G2
and/or G3 have one of the meanings mentioned above or, preferably,
G1 represents a C₆arylene or a saturated C₅-C₆cycloalkylene, or together with the group

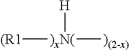

forms a heterocyclyl group comprising 5 or 6 ring atoms containing one nitrogen atom, or
together with the groups

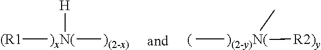

forms a heterocyclyl group comprising 5 or 6 ring atoms containing two nitrogen atoms; and/or
G2 represents a group selected from
   —CH₂—; —(CₙH₂ₙ)—; —(CₙH₂ₙ)—NH(C═O)NH—*; and
   —(CₙH₂ₙ)—[X—CₙH₂ₙ]ₘ—HN(C═O)NH—*,
   wherein
   X is —O— or —(NH)—,
   n is 2, and
   m is 0 or 1; and/or
G3 represents a C₆arylene; a saturated C₅-C₆cycloalkylene or together with the group

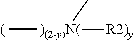

forms a heterocyclyl group comprising 5 or 6 ring atoms containing one nitrogen atom
wherein G1 and/or G3 may be unsubstituted or substituted, e.g. as described above.

Particularly preferred as compounds to be manufactured with the process according to the present invention are the compounds of formula (I), wherein the moiety:

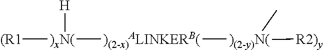

in formula (I) is selected from the following groups:

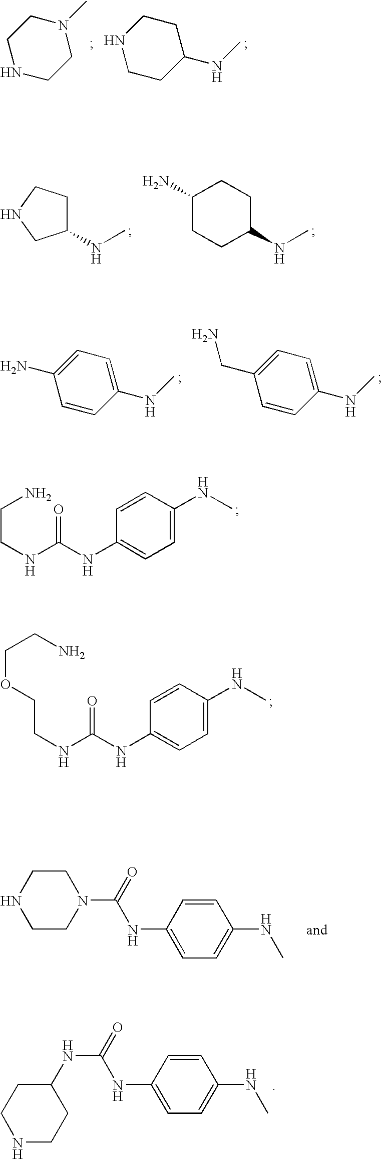

The compound of formula (II), (1S,5R)-7-oxo-2,6-diaza-bicyclo[3.2.0]heptane-6-sulfonic acid, can e.g. be prepared according to known methods, for example, as described in J. Med. Chem. 1988, 3961; EP 508 234 A2; J. Org. Chem. 1982, 5160 or WO 2007/065288 A2 starting from (1S,5R)-7-oxo-2,6-diaza-bicyclo[3.2.0]heptane-2-carboxylic acid tert.-butyl ester by reaction with the pyridine sulfur trioxide complex (Py*SO₃) followed by deprotection with TFA.

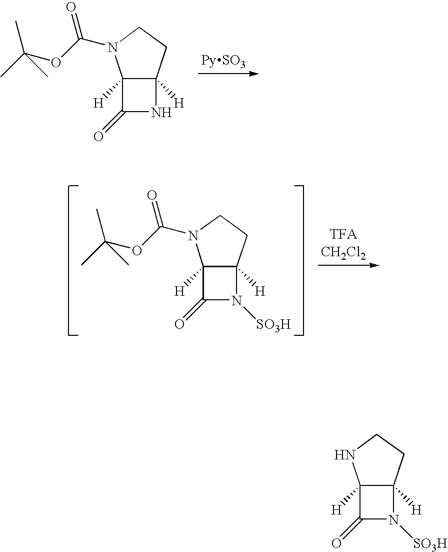

Suitable succinimidyl derivatives of formula (III) can e.g. be prepared either from commercially available or from synthesized amines, which are previously reacted with an amino protecting agent in order to introduce the amino protecting group Pr in the protected amine (VI), in the presence of N,N′-disuccinimidyl carbonate (VII) according to general procedures described in Tetrahedron 2001, 4311, Angew. Chem., Int. Ed. 2002, 1895 or J. Garb. Chem. 2003, 317 as shown in the following scheme:

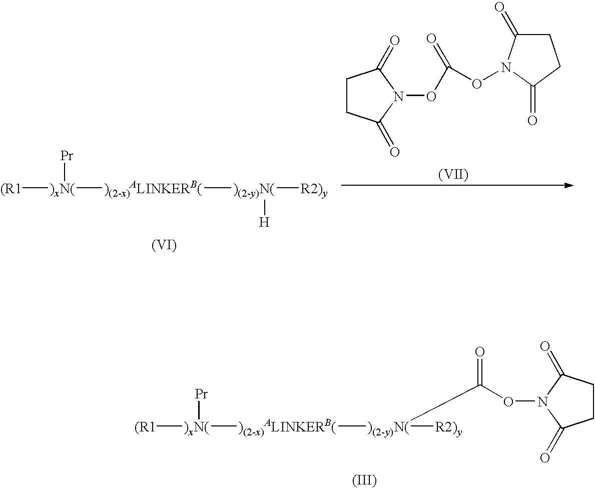

The amino protecting groups t-butyloxy carbonyl (t-Boc), 1-methyl-1-(4-biphenylyl)ethyloxy carbonyl (Bpoc), 1-(1-adamantyl)-1-methylethyloxy carbonyl (Adpoc), 1-(3,5-di-t-butylphenyl)-1-methylethyloxy carbonyl (t-Bumeoc), 1-adamantyloxy carbonyl (Adoc), p-methoxybenzyloxy carbonyl (Moz), and o,p-dimethoxybenzyloxy carbonyl, which are useful according to the invention, are acid labile. t-Butyloxy carbonyl (t-Boc) is preferred as amino protecting group.

For the purposes of the present invention “dipolar aprotic solvent” means a solvent with a sizable permanent dipole moment and a relative permittivity (or dielectric constant) greater than about 15 that cannot donate labile hydrogen atoms (are non-protogenic in a given situation) (IUPAC Compendium of Chemical Terminology 2nd Ed. 1997). Dipolar aprotic solvents include, for example, acetone; methyl ethyl ketone; acetonitrile; N,N-dimethylformamide (DMF) and dimethyl sulfoxide (DMSO), N-methyl-pyrrolidine (NMP), dimethylacetamide (DMA), and/or hexamethyl-phosphoramide (HMPA).

Preferably, the dipolar aprotic solvent exhibits a certain minimum solubility for the compound of formula (II), i.e. for (1S,5R)-7-oxo-2,6-diaza-bicyclo[3.2.0]heptane-6-sulfonic acid, preferably of at least 50 mg/mL, more preferably of at least 100 mg/mL, measured at 25° C. By way of example, DMSO can dissolve up to 200 mg/mL of (1S,5R)-7-oxo-2,6-diaza-bicyclo[3.2.0]heptane-6-sulfonic acid at 25° C.

The most preferred polar aprotic solvents for the purposes of the present invention are DMSO and DMF.

Suitable bases for use in the reaction step A include sodium carbonate, sodium hydrogenocarbonate, potassium carbonate, potassium hydrogencarbonate, cesium carbonate.

The reaction temperature for performing process step (A) is not particularly critical. Reaction temperatures and times can vary depending on the specific succinimidyl derivative used. Reaction step A is preferably performed at ambient or increased temperatures, preferably e.g. from about 15 to 100° C., more preferably from 20 to 85° C. Suitable reaction times range preferably form about 2 hours to 60 hours, in particular from 5 to 25 hours.

Although not necessary, as it is in case of using the unprotected succinimidyl derivatives for reaction with the (1S,5R)-7-oxo-2,6-diaza-bicyclo[3.2.0]heptane-6-sulfonic acid, it can, in certain cases, be of advantage to use a slight stoichiometric excess of the compound of formula (III), e.g. an excess of 5 to 10 mole percent, in order to accelerate process step (A) and to increase the yield. A significant reaction of the compound of formula (II) with itself, as found with the unprotected succinimidyl derivatives according to prior art, does not occur with the compounds of formula (III) so that the excess is not detrimental to the purity of the main product of said process step.

The compounds of formula (IV) obtained in process step (A) are preferably washed with a suitable solvent, e.g. ethyl acetate or acetone, and isolated by filtration.

The compound of formula (IV) is then dissolved in formic acid at ambient or, preferably, slightly reduced temperature. Suitable temperatures range from about 5 to 30° C., preferably from 10 to 15° C. A mixture of formic acid with hydrochloric acid, formic acid with hydrobromic acid, acetic acid with hydrochloric acid or acetic acid with hydrobromic acid can also be used. The mixing ratio is not particularly critical and can broadly vary.

Preferably after leaving the solution for some time, e.g. for 15 minutes to 5 hours, more preferably for 30 to 120 minutes, at such temperatures for a complete removal of the amine protecting group, the compound of formula (I) is isolated, preferably by precipitating of the compound of formula (I) from the acid solution with organic solvents. Suitable solvents are chosen depending on the particular compound of formula (I). Particularly suitable solvents for many compounds of formula (I) include acetone, acetonitrile, ethyl acetate and mixtures of such solvents. Although the obtained precipitate is in an amorphous state, the compounds of formula (I) obtained with the process of the present invention exhibit excellent purity.

The invention furthermore relates to compounds of formula (IV)

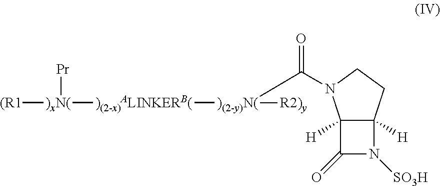

which are intermediates in the process according to the present invention and wherein Pr; ^{A}LINKER^{B}; R1; R2; x, y, (—) and, if groups of formula

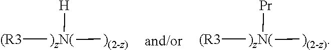

are present, R3 and z have one of the meaning as described above, with the exception of the compound of formula:

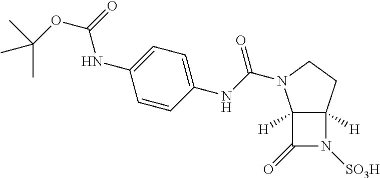

which is in different context known already from WO 2007/065288:

Particularly preferred are the aforementioned compounds wherein the moiety ^{A}LINKER^{B} is free from optional groups of the formulae:

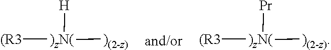

More particularly preferred are the compound of formula (IV), wherein the moiety:

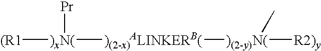

is selected from the following groups:

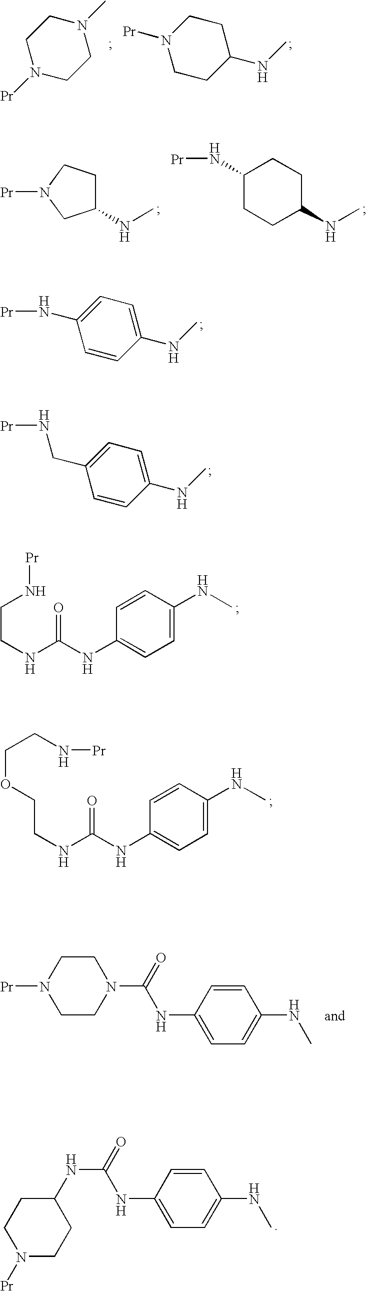

Also preferred are compounds of formula (IV), wherein Pr is t-butyloxy carbonyl (t-Boc).

### EXAMPLE 1

### (a) Preparation of (1S,5R)-2-[N-(4-{[(2-[((tert-butoxy)carbonylamino]ethyl)amino]-carbonylamino}phenyl)carbamoyl]-7-oxo-2,6-diazabicyclo[3.2.0]heptane-6-sulfonic acid

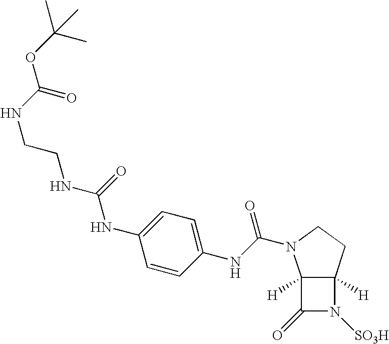

To a solution of dimethylsulfoxyde (13 mL) containing (1S,5R)-7-oxo-2,6-diazabicyclo[3.2.0]heptane-6-sulfonic acid (1 g, 5.2 mmol) is added at room temperature sodium hydrogen carbonate (0.437 g, 5.2 mmol) and then ({2-[(tert-butoxy)carbonylamino]ethyl}amino)-N-{4-[(2,5-dioxoazolidinyloxy)carbonylamino]-phenyl}carboxamide (WO2007/065288) (2.266 g, 5.2 mmol). The resulting mixture is stirred for 24 hours at room temperature. Sodium hydrogenocarbonate (0.044 g, 0.52 mmol) is added and the mixture is stirred at room temperature for an additional 1 hour. Insoluble material is filtrated off and the filtrate is concentrated to dryness in vacuo. The oily residue is triturated in ethyl acetate (100 mL) for 1 hour at room temperature. The light grey precipitate is filtered and the collected substance is washed with ethyl acetate (3×20 mL). The solid is dried under high vacuum to yield 2.56 g of the expected compound.

¹H NMR (d₆-DMSO): δ 1.38 (s, 9H), 1.64 (m, 1H), 2.29 (dd, 2H), 2.99 (m. 2H), 3.12 (m, 2H), 3.18 (m, 1H), 3.97 (dd, 1H), 4.38 (t, 1H), 5.21 (d, 1H), 6.08 (t, 1H), 6.84 (t, 1H), 7.20-7.35 (m, 4H), 8.35 (s, 1H), 8.37 (s, 1H)

This procedure is used for the preparation of the further examples in Table 1. Reaction temperatures and times are also indicated in Table 1.

TABLE 1

| | | | Reaction | Reaction |
|---|---|---|---|---|
| No. | Compound of formula (III) | Compound of formula (IV) | Temp. | Temp. |
| 1(b) | 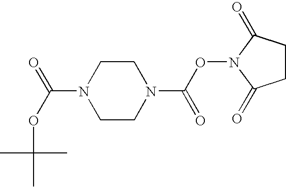 | 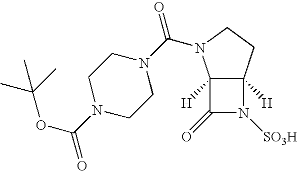 | 85° C. | 48 h |
| 1(c) | 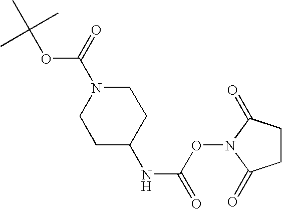 | 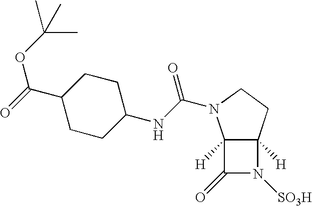 | 75° C. | 5 h |
| 1(d) | 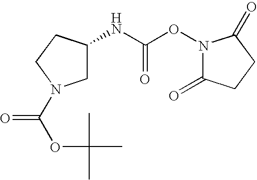 | 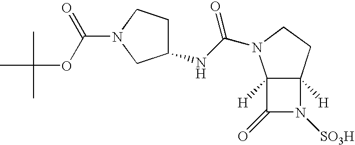 | 75° C. | 5 h |
| 1(e) | 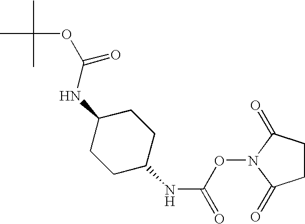 | 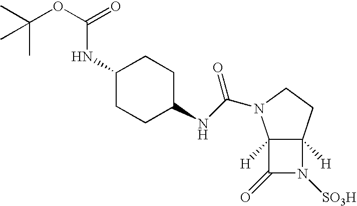 | 75° C. | 5 h |
| 1(f) | 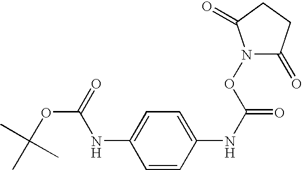 | 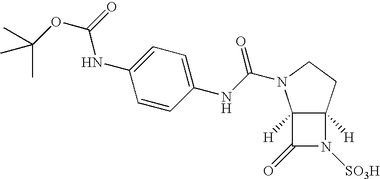 | Room Temperature | Overnight |
| 1(g) | 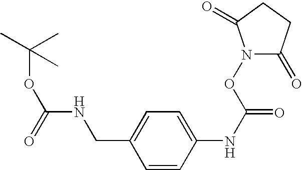 | 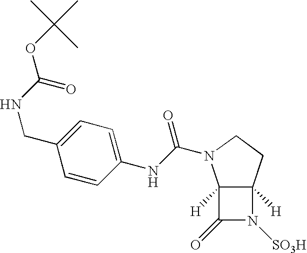 | Room Temperature | Overnight |

DMF may substitute DMSO as the solvent in all aforementioned Examples.

### EXAMPLE 2

### (a) Preparation of (1S,5R)-2-[N-(4-{[(2-aminoethyl)amino]carbonylamino}phenyl) carbamoyl]-7-oxo-2,6-diazabicyclo[3.2.0]heptane-6-sulfonic acid

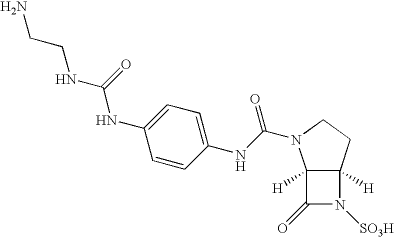

(1S,5R)-2-[N-(4-{[(2-[((tert-butoxy)carbonylamino]ethyl)amino]carbonyl amino}phenyl)carbamoyl]-7-oxo-2,6-diazabicyclo[3.2.0]heptane-6-sulfonic acid (2 g, 3.9 mmol) is dissolved in formic acid (10 mL) and the light red solution is stirred for 1 h at 10-15° C. Then acetone (100 mL) is added. The resulting precipitate is collected by filtration and washed with acetone (3×25 mL) to afford 1.3 g of the desired compound as amorphous material. The ¹H NMR (d₆-DMSO) data correspond to those disclosed for compound 324 in WO 2007/065288 A2 (cf. Example 43 thereof).

This procedure is also used for the Examples described in the Table 2.

TABLE 2

| No. | Compound of formula (I) | Work up | ¹H NMR (d₆-DMSO) |
|---|---|---|---|
| 2(b) | 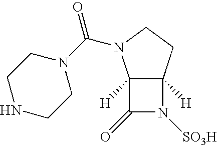 | Acetone | 1.40-1.60 (m, 1H); 2.15-2.25 (m, 1H); 3.00-3.50 (m, 9H); 3.90-4.05 (m, 1H); 4.38 (m, 1H); 5.05 (d, J = 4.3, 1H); 9.20-9.60 (br, 1H). |
| 2(c) | 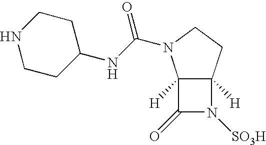 | Acetone | 1.55-1.65 (m, 3H); 1.83-1.93 (m, 2H); 2.22-2.32 (m, 1H); 2.92-3.10 (m, 3H); 3.20-3.30 (m, 1H); 3.64-3.74 (m, 1H); 3.82 (m, 1H); 4.34 (m, 1H); 5.06 (d, J = 4.3, 1H); 6.46 (d, J = 5.4, 1H); 8.28 (br, 1H). |
| 2(d) | 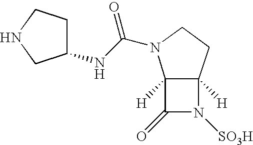 | Acetone | 1.50-1.60 (m, 1H); 1.85-1.95 (m, 1H); 2.20-2.25 (m, 1H); 3.00-3.50 (m, 7H); 3.84 (m, 1H); 4.14-4.24 (m, 1H); 4.36 (t, J = 4.7, 1H); 5.03 (d, J = 4.3, 1H); 6.69 (d, J = 5.4, 1H); 8.76 (br, 1H). |
| 2(e) | 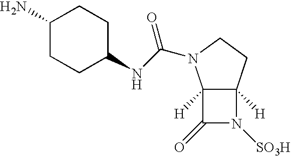 | Acetone | 1.20-1.40 (m, 4H); 1.50-1.60 (m, 1H); 1.77-1.97 (m, 4H); 2.20-2.25 (m, 1H); 2.95-3.05 (m, 2H); 3.16-3.21 (m, 1H); 3.81 (m, 1H); 4.32 (m, 1H); 5.02 (d, J =4.3, 1H); 6.24 (d, J = 7.6, 1H); 7.71 (br, 3H). |
| 2(f) | 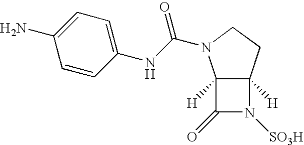 | Acetone/ Ethyl acetate (1:1) | 1.61-1.71 (m, 1H); 2.29-2.34 (m, 1H); 3.15-3.23 (m, 1H); 3.95-4.01 (m, 1H); 4.41 (m, 1H); 5.23 (d, J = 4.3, 1H); 7.08 (d, J = 9.1, 2H); 7.49 (d, J = 9.1, 2H) 8.60 (s, 1H). |
| 2(g) | 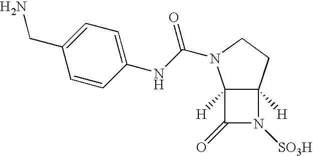 | Acetone/ Ethyl acetate (1:1) | 1.63-1.73 (m, 1H); 2.28-2.38 (m, 1H); 3.15-3.25 (m, 1H); 3.89-4.05 (m, 3H); 4.42 (m, 1H); 5.26 (d, J = 4.3, 1H); 7.35 (m, 2H); 7.55 (m, 2H); 8.65 (s, 1H). |

Solvents other than acetone or acetone/ethyl acetate (1:1) can also be used, e.g. acetonitrile and mixtures thereof with acetone and/or ethyl acetate for precipitating the compounds of formula (I).

1. A process for the production of a compound of formula (I) 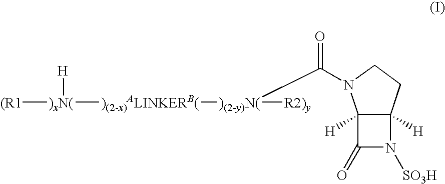
wherein
^{A}LINKER^{B} represents a linker moiety of formula
^{A}[G1-G2*-G3]^{B} (V)
wherein
^{A} and ^{B} indicate the orientation of the group of formula (V) in formula (I);
G1, G2 or G3 may be present or absent, with the proviso that at least one of G1 or G3 is present; and,
G1 if x is 1 and y is 1 and both, G2 and G3, are absent or if x is 1 and G2 and/or G3 are present, represents a C₆-C₁₄arylene; a saturated or non-aromatic unsaturated C₃-C₉cycloalkylene or a saturated or unsaturated heterocyclodiyl group comprising 5 to 9 ring atoms containing one or more heteroatoms selected from N, O and S, which groups may be unsubstituted or substituted or, if x is 0 and y is 1 and both, G2 and G3, are absent or if x is 0 and G2 and/or G3 are present, together with the group 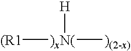
forms a heterocyclyl group comprising 5 to 9 ring atoms containing one or more heteroatoms selected from N, O and S, which may be unsubstituted or substituted; or,
if x is 0 and y is 0 and both, G2 and G3, are absent, together with the groups 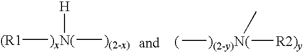
forms a heterocyclyl group comprising 5 to 9 ring atoms containing two or more heteroatoms selected from N, O and S, which may be unsubstituted or substituted;
G2 represents a group selected from
—(CₙH₂ₙ—)—; —(NH)—; —(C═O)—; —(C═O)NH—*; —HN(C═O)—*;
—HN(C═O)NH—; —(CₙH₂ₙ)—[X—CₙH₂ₙ]ₘ—(C═O)NH—*;
—HN(C═O)—[CₙH₂ₙ]—X]ₘ—(CₙH₂ₙ)—*;
—(CₙH₂ₙ)—[X—CₙH₂ₙ]ₘ—HN(C═O)—*;
—(O═C)NH—[CₙH₂ₙ]—X]ₘ—(CₙH₂ₙ)—*;
—(CₙH₂ₙ)—[X—CₙH₂ₙ]m—HN(C═O)NH—*, and
—HN(C═O)NH—(CₙH₂ₙ)—[X—CₙH₂ₙ]ₘ—*
wherein
* indicates the bond of G2 indicated in formula (V) with * and X represents, independently at each occurrence,
—O—; —NH—; —(C═O)NH—; —NH(C═O)—; —(C═O)O—; —O(C═O)—;
—NH(C═O)NH—; —NH(C═O)O— or —O(C═O)NH—
n is, independently at each occurrence, 1, 2, 3 or 4, in particular 2; and
m is 0, 1, 2 or 3, in particular 0 or 1; and,
G3 if y is 1 and G1 and/or G2 are present, represents a C₆-C₁₄arylene; a saturated or non-aromatic unsaturated C₃-C₉cycloalkylene or a saturated or unsaturated heterocyclodiyl group comprising 5 to 9 ring atoms containing one or more heteroatoms selected from N, O and S, which groups may be unsubstituted or substituted, or,
if y is 0 and x is 1 and both, G1 and G2, are absent or if y is 0 and G1 and/or G2 are present, together with the group 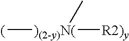
forms a heterocyclyl group comprising 5 to 9 ring atoms containing one or more heteroatoms selected from N, O and S, which may be unsubstituted or substituted,
which linker group may furthermore optionally contain one or more groups of formula: 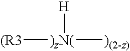
and/or other substituents;
R1 represents hydrogen or a C₁-C₄-alkyl group;
R2 represents hydrogen or a C₁-C₄-alkyl group;
R3 independently at each occurrence, represents hydrogen or a C₁-C₄-alkyl group;
x is 0 or 1;
y is 0 or 1;
z independently at each occurrence, is 0 or 1; and
(—) represents a single bond between a primary, secondary or tertiary carbon atom of the moiety ^{A}LINKER^{B} and the adjacent nitrogen atom,
in which process
(A) a compound of formula (II) 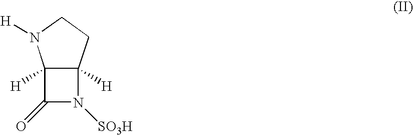
is reacted with a compound of formula (III) 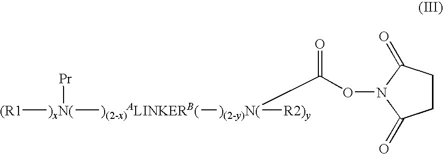
wherein
Pr represents an amino protecting group selected from t-butyloxy carbonyl (t-Boc), 1-methyl-1-(4-biphenyl)ethyloxy carbonyl (Bpoc), 1-(1-adamantyl)-1-methylethyloxy carbonyl (Adpoc), 1-(3,5-di-t-butylphenyl)-1-methylethyloxy carbonyl (t-Bumeoc), 1-adamantyloxy carbonyl (Adoc), p-methoxybenzyloxy carbonyl (Moz) and o,p-dimethoxybenzyloxy carbonyl;
^{A}LINKER^{B} has the same meaning as in formula (I) with the exception that one or more of the optional groups of formula: 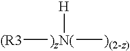
may be replaced by a group of formula: 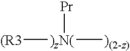
and
R1; R2; R3; x; y; z and (—), at each occurrence, have the same meaning as in formula (I) and Pr is as defined above;
in a dipolar aprotic solvent in the presence of a base to obtain a compound of formula (IV) 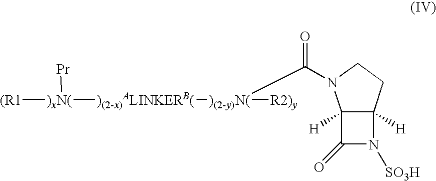
wherein
Pr; ^{A}LINKER^{B}; R1; R2; R3; x; y; z; and (—), at each occurrence, have the same meaning as in formula (III);
which compound is then
(B) deprotected by reaction with formic acid or a mixture of formic acid or acetic acid with hydrochloric acid or hydrobromic acid, to give the compound of formula (I).

2. A process according to claim 1 for the production of a compound of formula (I), wherein
^{A}LINKER^{B} represents a linker moiety of formula
^{A}[G1-G2*-G3]^{B} (V)
wherein
^{A} and ^{B} indicate the orientation of the group of formula (V) in formula (I);
G1, G2 or G3 may be present or absent, with the proviso that at least one of G1 or G3 is present; and,
G1 if x is 1 and y is 1 and both, G2 and G3, are absent or if x is 1 and G2 and/or G3 are present, represents a C₆-C₁₄arylene; a saturated or non-aromatic unsaturated C₃-C₇cycloalkylene or a saturated or unsaturated heterocyclodiyl group comprising 5 to 7 ring atoms containing one or more heteroatoms selected from N, O and S, which groups may be unsubstituted or substituted or,
if x is 0 and y is 1 and both, G2 and G3, are absent or if x is 0 and G2 and/or G3 are present, together with the group 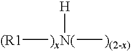
forms a heterocyclyl group comprising 5 to 7 ring atoms containing one or more heteroatoms selected from N, O and S, which may be unsubstituted or substituted; or,
if x is 0 and y is 0 and both, G2 and G3, are absent, together with the groups 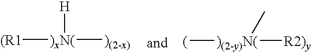
forms a heterocyclyl group comprising 5 to 7 ring atoms containing two or more heteroatoms selected from N, O and S, which may be unsubstituted or substituted;
G2 represents a group selected from
—(CₙH₂ₙ)—; —(NH)—; —(C═O)—; —(C═O)NH—*; —HN(C═O)—*;
—HN(C═O)NH—; —(CₙH₂ₙ)—[X—CₙH₂ₙ]ₘ—(C═O)NH—*
—(CₙH₂ₙ)—[X—CₙH₂ₙ]ₘ—HN(C═O)—*, and
—(CₙH₂ₙ)—[X—CₙH₂ₙ]ₘ—HN(C═O)NH—*,
wherein
* indicates the bond of G2 indicated in formula (V) with * and X represents, independently at each occurrence,
—O—; —NH—; —(C═O)NH—; —NH(C═O)—; —(C═O)O—; —O(C═O)—;
—NH(C═O)NH—; —NH(C═O)O— or —O(C═O)NH—
n is, independently at each occurrence, 1, 2, 3 or 4, in particular 2; and
m is 0, 1, 2 or 3, in particular 0 or 1; and,
G3 if y is 1 and G1 and/or G2 are present, represents a C₆-C₁₄arylene; a saturated or non-aromatic unsaturated C₃-C₇cycloalkylene or a saturated or unsaturated heterocyclodiyl group comprising 5 to 7 ring atoms containing one or more heteroatoms selected from N, O and S, which groups may be unsubstituted or substituted, or,
if y is 0 and x is 1 and both, G1 and G2, are absent or if y is 0 and G1 and/or G2 are present, together with the group 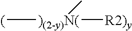
forms a heterocyclyl group comprising 5 to 7 ring atoms containing one or more heteroatoms selected from N, O and S, which may be unsubstituted or substituted,
which linker group may furthermore optionally contain one or more groups of formula: 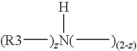
and/or other substituents.

3. A process according to claim 1 for the production of a compound of formula (I), wherein ^{A}LINKER^{B} is free from optional groups of formula:

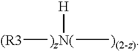

4. A process according to claim 1, wherein
^{A}LINKER^{B} in formula (I) represents a moiety of formula
[G1] and x is 0 or 1 and y is 1, or x and y are both 0;
[G3] and y is 0 or 1;
^{A}[G2*-G3]^{B} and x and y are both 1; or
A[G1-G2*-G3]B and x and y are, independently of one another, 0 or 1, and
G1, G2 and or G3 have one of the meanings given in claim 1, 2 or 3.

5. A process according to any one of claim 1, wherein
G1 represents a C₆arylene or a saturated C₅-C₆cycloalkylene, or together with the group 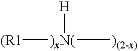
forms a heterocyclyl group comprising 5 or 6 ring atoms and containing one nitrogen atom, or
together with the groups 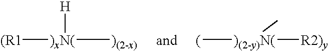
forms a heterocyclyl group comprising 5 or 6 ring atoms containing two nitrogen atoms;
G2 represents a group selected from
—CH₂—; —(CₙH₂ₙ)—NH(C═O)NH—*; and
—(CₙH₂ₙ)—[X—CₙH₂ₙ]ₘ—HN(C═O)NH—*,
wherein X is —O— or —(NH)—, and
n is 2; and
G3 represents a C₆arylene; a saturated C₅-C₆cycloalkylene or together with the group 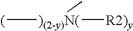
forms a heterocyclyl group comprising 5 or 6 ring atoms containing one nitrogen atom
wherein G1 and/or G3 may be unsubstituted or substituted.

6. A process according to claim 1, wherein y is 1.

7. A process according to claim 6, wherein R2 is hydrogen.

8. A process according to claim 1, wherein R1 represents hydrogen.

9. A process according to claim 1, wherein the moiety: 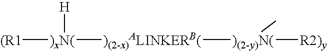
in formula (I) is selected from the following groups:

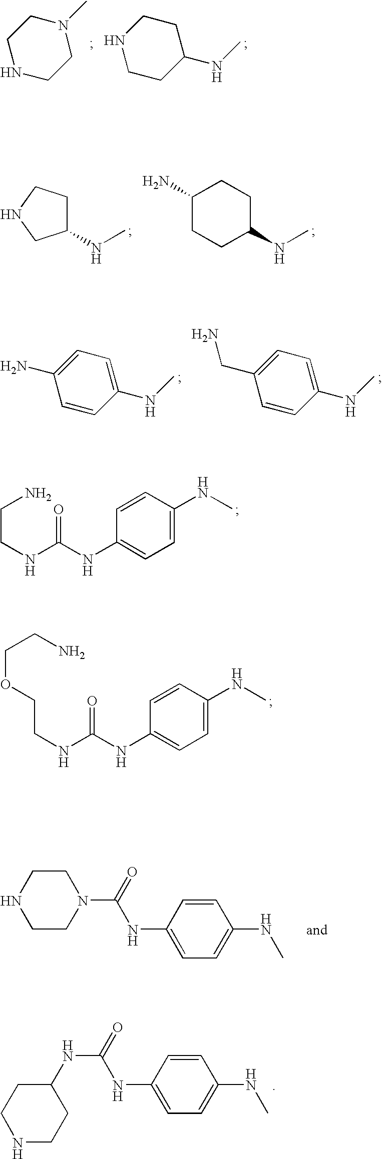

10. A process according to claim 1, wherein the concentration of the compound of formula (II) in said dipolar aprotic solvent in step A is at least 50 mg/mL, in particular from 50 mg/mL to 150 mg/mL, measured at 25° C.

11. A process according to anyone claim 1, wherein said dipolar aprotic solvent is DMF, DMSO or a mixture thereof.

12. A process according to claim 1, wherein the reaction temperature in process step (A) is from 15 to 100° C., preferably from 20 to 85° C., most preferably about ambient temperatures, like in particular 15 to 35° C.

13. A process according to claim 1, wherein the reaction time in process step (A) is from 5 to 60 h, preferably from 5 to 25 h.

14. A process according to claim 1, wherein the compound of formula (IV) is isolated as an intermediate and is, optionally, purified by washing it with a solvent before using it for process step (B).

15. A process according to claim 1, wherein in process step (B) the compound of formula (IV) is dissolved in formic acid at a temperature of 5 to 20° C., preferably 10 to 15° C.

16. A process according to claim 15, wherein the solution of the compound of formula (IV) in formic acid is held at said temperatures for 15 minutes to 5 hours.

17. A process according to claim 1, wherein the compound of formula (I) is isolated in process step (B) by precipitating it with a solvent selected from acetone, ethyl acetate and mixtures thereof or acetonitrile and mixtures thereof with acetone and/or ethyl acetate.

18. A process according to claim 1, wherein in process step (B) a mixture of formic acid with hydrochloric acid is used instead of formic acid.

19. A compound of formula (IV) 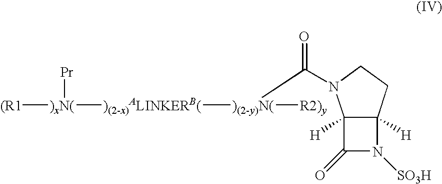
wherein
Pr represents a group selected from t-butyloxy carbonyl (t-Boc), 1-methyl-1-(4-biphenylyl)ethyloxy carbonyl (Bpoc), 1-(1-adamantyl)-1-methylethyloxy carbonyl (Adpoc), 1-(3,5-di-t-butylphenyl)-1-methylethyloxy carbonyl (t-Bumeoc), 1-adamantyloxy carbonyl (Adoc), p-methoxybenzyloxy carbonyl (Moz), o,p-dimethoxybenzyloxy carbonyl;
^{A}LINKER^{B} represents a linker moiety of formula
^{A}[G1-G2*-G3]^{B} (V)
wherein
^{A} and ^{B} indicate the orientation of the group in formula (I);
G1, G2 or G3 may be present or absent, with the proviso that at least one of G1 or G3 is present; and,
G1 if x is 1 and y is 1 and both, G2 and G3, are absent or if x is 1 and G2 and/or G3 are present, represents a C₆-C₁₄arylene; a saturated or non-aromatic unsaturated C₃-C₉cycloalkylene, preferably C₃-C₇cycloalkylene, or a saturated or unsaturated heterocyclodiyl group comprising 5 to 9 ring atoms, preferably 5 to 7 ring atoms, containing one or more heteroatoms selected from N, O and S, which groups may be unsubstituted or substituted or,
if x is 0 and y is 1 and both, G2 and G3, are absent or if x is 0 and G2 and/or G3 are present, together with the group 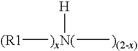
forms a heterocyclyl group comprising 5 to 9, preferably 5 to 7, ring atoms containing one or more heteroatoms selected from N, O and S, which may be unsubstituted or substituted; or,
if x is 0 and y is 0 and both, G2 and G3, are absent, together with the groups 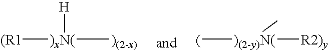
forms a heterocyclyl group comprising 5 to 9, preferably 5 to 7, ring atoms containing two or more heteroatoms selected from N, O and S, which may be unsubstituted or substituted;
G2 represents a group selected from
—(CₙH₂ₙ)—; —(NH)—; —(C═O)—; —(C═O)NH—*; —HN(C═O)—*;
—HN(C═O)NH—; —(CₙH₂ₙ)—[X—CₙH₂ₙ]ₘ—(C═O)NH—*;
—HN(C═O)—[CₙH₂ₙ]—X]ₘ—(CₙH₂ₙ)—*;
—(CₙH₂ₙ)—[X—CₙH₂ₙ]ₘ—HN(C═O)—*;
—(O═C)NH—[CₙH₂ₙ]—X]ₘ—(CₙH₂ₙ)—*;
—(CₙH₂ₙ)—[X—CₙH₂ₙ]—HN(C═O)NH—*, and
—HN(C═O)NH—(CₙH₂ₙ)—[X—CₙH₂ₙ]ₘ—*
wherein
* indicates the bond of G2 indicated in formula (V) with * and X represents, independently at each occurrence,
—O—; —NH—; —(C═O)NH—; —NH(C═O)—; —(C═O)O—; —O(C═O)—;
—NH(C═O)NH—; —NH(C═O)O— or —O(C═O)NH—
n is, independently at each occurrence, 1, 2, 3 or 4, in particular 2; and
m is o, 1, 2 or 3, in particular o or i; and,
G3 if y is 1 and G1 and/or G2 are present, represents a C₆-C₁₄arylene; a saturated or non-aromatic unsaturated C₃-C₉cycloalkylene, preferably a C₃-C₇cycloalkylene or a saturated or unsaturated heterocyclodiyl group comprising 5 to 9, preferably 5 to 7 ring atoms containing one or more heteroatoms selected from N, O and S, which groups may be unsubstituted or substituted, or,
if y is 0 and x is 1 and both, G1 and G2, are absent or if y is 0 and G1 and/or G2 are present, together with the group 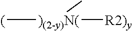
forms a heterocyclyl group comprising 5 to 9, preferably 5 to 7 ring atoms containing one or more heteroatoms selected from N, O and S, which may be unsubstituted or substituted,
which linker group may furthermore optionally contain one or more groups of formula: 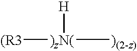
and/or other substituents;
R1 represents hydrogen or a C₁-C₄-alkyl group;
R2 represents hydrogen or a C₁-C₄-alkyl group;
R3 independently at each occurrence, represents hydrogen or a C₁-C₄-alkyl group;
x is 0 or 1;
y is 0 or 1;
z independently at each occurrence, is 0 or 1; and
(—) represents a single bond between a primary, secondary or tertiary carbon atom of the linker moiety and the adjacent nitrogen atom;
with the exception of the compound of formula:

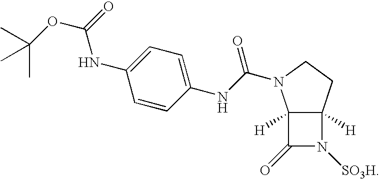

20. A compound according to claim 19, wherein the moiety ^{A}LINKER^{B} is free from optional groups of the formulae:

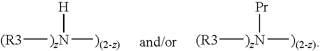

21. A compound according to claim 19, wherein ^{A}LINKER^{B}, R1, R2, x and/or y are as defined in any one of claims 4 to 8.

22. A compound according to claim 19, wherein the moiety: 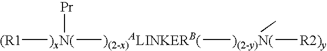
in formula (IV) is selected from the following groups:

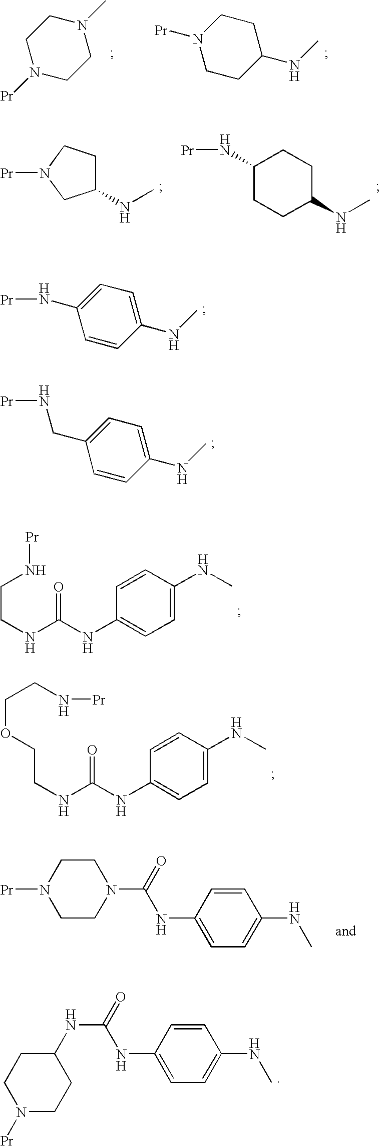

23. A compound according to claim 19, wherein Pr is t-butyloxy carbonyl (t-Boc).
